(19) United States
(12) Patent Application Publication
(10) Pub. No.: US 2009/0164001 A1
(43) Pub. Date: Jun. 25, 2009

(54) Socket For Fenestrated Tubular Prosthesis

(76) Inventors: DAVID P. BIGGS, Bloomington, IN (US); JICHAO SUN, West Lafayette, IN (US); ALAN R. LEEWOOD, Lafayette, IN (US)

Correspondence Address: BRINKS HOFER GILSON & LIONE/CHICAGO/COOK, PO BOX 10395, CHICAGO, IL, 60610, US

(21) Appl. No.: 12/233,359

(22) Filed: Sep. 18, 2008

Related U.S. Application Data
(60) Provisional application No. 61/008,947, filed on Dec. 21, 2007.

(51) Int. Cl. A61F 2/06

(52) U.S. Cl. 623/1.35; 623/1.15; 623/1.13

(57) ABSTRACT

A stent graft adapted to telescopically receive a secondary stent graft having an evertible, elastic socket communicating with an opening in the stent graft. The socket comprises an elastic wall that forms a lumen with a stent at least partially encased within the wall. The socket may be adapted for use with stent grafts for implantantation in an aneurysm.

### CROSS-REFERENCE TO RELATED APPLICATION

The present application claims priority to Provisional Application Ser. No. 61/008,947, filed Dec. 21, 2007, which is a continuation-in-part of the co-pending application claiming priority to Application Ser. No. 60/962,109, filed Jul. 26, 2007.

### TECHNICAL FIELD

This invention relates to a medical device for implantation within the human or animal body for the treatment or repair of aortic aneurysms.

### BACKGROUND

One of the primary functions of the fenestrated stent graft with bridging stent is to maintain patency of the renal arteries even though the proximal end of the stent-graft extends beyond the renal arteries. Conventionally, a balloon expandable bare stent is deployed into the renal arteries through the fenestration in the main graft to assure alignment is maintained while the stent-graft is being delivered (e.g., manipulated) and continues to maintain patency post-procedure. Fenestrated stent grafts usually use a sutured reinforcement ring, for example a nitinol ring, with some type of imageable markers (see FIG. 1). The distal part of the metal stent is deployed into the renal artery and the proximal end is held against the graft via the sutured nitinol ring to ensure a secure fixation.

Since the arterial tree is constantly under pulsatile motion due to hemodynamic and anatomical loads, the deployed bare metal stent is very often under severe and complicated loading conditions (bending, radial pulsation, shearing, etc.) This must be borne entirely through the narrow interface presented by the ring. Furthermore, there is normally considerable plastic deformation induced to the stent during current deployment techniques which can lead to localized fracture of the stent that negates the alignment function of the fenestration stent.

When an aneurysm extends infra-renally, a covered stent may be needed to bridge this aneurysm so that the blood flow is maintained to the kidneys. In such cases, the interface between the fenestrated stent graft and the infra-renally placed covered stent must, in addition to providing alignment, provide a hemodynamic seal in a very dynamic environment. The difficulty in providing adequate renal support using either covered or bare metal stents is the narrow interaction zone between the infra-renally placed stent and the fenestrated stent graft. The infra-renally placed stent must handle the stresses caused by the pulsatile blood flow created by the heart.

One of the major functional requirements for an iliac branch vessel device bridging a covered stent is sealing and basic attachment. In order to achieve an effective seal at the proximal end where a covered stent fits into a bifurcated graft, devices in the art utilize two rings with a fixed diameter and a flexible stent with a nominal diameter less than the fixed diameter of the two rings. However, due to the relative rigidity of the fixed diameter rings and the inextensibility of the graft, the diameter of the covered bridging socket may not exceed about a millimeter over the fixed diameter of the rings. The resultant nature of this socket system can restrict the proximal end of the bridging stent to a diameter less than the distal landing zone in the common iliac in some instances. Thus, the result is a stent that may non-uniformly taper along its axis as the bridging stent transitions out of the branch vessel device socket.

This raises two important issues: the effectiveness of the seal across the wide range of vessel sizes and the potential fatigue problems while undergoing pulsatile loading aggravated by the taper. A dramatic taper can potentially cause damaging plastic deformation and nonuniform loading on the covered balloon expandable stent, especially within the transition region outside of the ring socket, which may greatly shorten stent durability or even tear or pinch the covering.

Further, since the rings essentially create a fixed diameter socket, it will not accommodate the recoil of a covered stent. Therefore, for some covered stents with a large recoil rate, the sealing function may be problematic.

Thus, a need exists for a socket for use with an endoluminal prosthesis which will minimize or eliminate the fatigue suffered by infra-renally placed stents. This would enable graft systems extending into renal arteries or other branched vessels to be safely utilized in patients for long periods of time without concern of premature failure due to wear. Such sockets need a high pulsatile fatigue life. Pulsatile fatigue is the fatigue resistance of the stent to pulsing radial loads, such as blood pressure loads. In practice, pulsatile fatigue is tested by expanding a stent into a flexible tube that is then filled with a fluid and pulsed rapidly to alter the diameter of the stent cyclically. Thus, a need exists for a prosthetic endovascular graft system which incorporates sockets that are designed to minimize cyclic stresses and thus avoid fatigue failure.

### BRIEF SUMMARY

The present invention provides a stent graft for endoluminal implantation. The stent graft is adapted to telescopically receive a secondary stent graft and is characterized in that the stent graft comprises at least one socket communicating with at least one opening in the stent graft. The at least one socket comprises an elastic wall that forms a lumen with a stent at least partially encased within the wall.

In one example, the stent graft is bifurcated with two distal openings and is adapted to telescopically receive a secondary stent graft. In another example, the socket forms a branch of the stent graft for telescopically receiving a secondary stent graft extending into an iliac artery. In yet another example, the socket is proximal to the bifurcation and comprises an elastic wall forming a lumen with a stent at least partially encased within the wall. Additionally, the stent graft may include a second socket in communication with a second opening in the stent graft.

In one aspect of the present invention, the stent graft is adapted to telescopically receive a secondary stent graft extending into a renal artery. In yet another aspect, the proximal end of the socket flares around the external or internal side of the wall opening. The socket has an expandable diameter that adjusts to the dynamic movement of the human body. In some examples, the socket is tapered, comprises reinforcing elements, or radiopaque markers. The reinforcing elements comprise nitinol or polyethylene fibers. The socket can extend radially from the tubular prosthesis at an acute, right, or obtuse angle. There are also examples where the socket is attached to the tubular prosthesis by gluing, stitching, repolymerization, dipping, casting, or is thermoformed.

In yet another aspect of the present invention, the socket may be made from polyurethane, expanded polytetratfluoroethylene (ePTFE), or any other polymer that provides sufficient elasticity, deformability, and biocompatibility. Reinforcing elements, such as nitinol or PET fibers, may be imbedded in the socket to adjust the radial and longitudinal stiffness. Radiopaque markers, such as gold, may be placed within the socket to assist in placement of the socket.

In general, the stent grafts of the present invention provide sockets that have a high degree of expanded radial stiffness and flexibility which may be used for long periods of time in a pulsatile environment without causing fatigue and fracture of the socket or overall prosthesis. The sockets are highly torsional and distendable while bridging the tubular prosthesis and/or the structural prosthesis in the target vessel.

In yet another example of the present invention, the present invention provides a tubular graft having a proximal end, a distal end, a lumen therethrough. There is an opening between the proximal end and distal end and an evertible, elastic socket disposed within the lumen of the tubular graft. The elastic socket comprising a socket wall, a first end, a second end, and a lumen therethrough, an everted configuration and extended configuration. The first end of the socket is disposed adjacent the opening of the tubular graft in the everted and extended configurations. The second end is disposed in the lumen of the graft in the everted configuration and is disposed external to the graft in the expanded configuration.

Accordingly, the present invention also provides a method for delivering such a prosthesis. The method comprises placing the tubular graft into a vessel having a branch artery; aligning the opening with the branch artery; and actuating the guidewire to evert the elastic socket and to deploy the elastic socket into the branch artery.

### BRIEF DESCRIPTION OF THE DRAWINGS

FIG. 1 shows a bare stent protruding from a fenestrated stent graft.

FIG. 2 shows a socket attached to the wall and around the opening of a fenestrated stent graft.

FIG. 3 shows a socket with a secondary stent telescopically placed within the socket.

FIG. 4 shows a proximal end of a stent graft with a socket in communication with an opening in the stent graft.

FIG. 5 shows a bifurcated stent graft with a socket portion in communication with a branch opening.

FIG. 6 shows a bifurcated stent graft with a socket with a secondary stent graft implanted into the socket.

FIGS. 7A through 7D are cross-sectional views of various sockets.

FIG. 8 is a cut-away view of an abdominal aortic aneurysm with a stent graft of the present invention implanted in the aorta with sockets bridging secondary stent grafts implanted in the renal arteries.

FIG. 9 is a cut-away view of an abdominal aortic aneurysm with a stent graft of the present invention implanted in the aorta with a socket implanted into the iliac artery.

FIG. 10 depicts a branched prosthesis implanted in the aortic arch with sockets extending into branch arteries with one socket receiving a secondary stent graft.

FIG. 11 a cross-sectional view of a stent graft with a socket in its initial position in the lumen of the stent graft.

FIG. 12A is a cross-sectional view of an abdominal aortic aneurysm with a stent graft implanted in the aorta. The socket is shown connected to a guidewire.

FIG. 12B is a cross-sectional view of the elastic socket deployed in the branch artery.

### DETAILED DESCRIPTION OF THE DRAWINGS AND THE PREFERRED EMBODIMENTS

Unless defined otherwise, all technical and scientific terms used herein have the same meaning as commonly understood to one of ordinary skill in the art to which this invention belongs.

Throughout this specification, when discussing the application of this invention to the aorta, the term distal, with respect to a prosthesis, is intended to refer to the end of the prosthesis furthest away in the direction of blood flow from the heart, and the term proximal is intended to mean the end of the prosthesis that, when implanted, would be nearest to the heart.

The term “graft or graft material” means a generally cannular or tubular member which acts as an artificial vessel or prosthesis. A graft by itself or with the addition of other elements, such as structural components, may be an endoluminal prosthesis. The graft comprises a single material, a blend of materials, a weave, a laminate, or a composite of two or more materials. The graft can also comprise polymer material that may be layered onto the mandrel of the present invention. Preferably, polymers of the present invention, although added in layers onto the mandrel, after curing, result in one layer that encapsulates a stent or woven graft. This also aids in decreasing the incidence of delamination of the resulting endovascular prosthesis.

The graft material is a biocompatible material that is both flexible and abrasion resistant. Furthermore, the graft material should be selected from those materials that are particularly well suited for bonding with polymer. Preferably, the graft material is a woven polyester. More preferably, the graft material is a polyethylene terephthalate (PET), such as DACRON® (DUPONT, Wilmington, Del.) or TWILLWEAVE MICREL® (VASCUTEK, Renfrewshire, Scotland). Woven polyesters, such as Dacron, possess varying degrees of porosity, where the degree of porosity may be selectively controlled based on the weaving or knitting process that is used to produce the woven polyester. Consequently, depending on the application, the porosity may be adjusted to encourage incorporation of a patient's tissue into the woven graft material, which in turn may more securely anchor the prosthesis within the patient's vessel or lumen. Furthermore, the degree of porosity can also be adjusted to provide a woven graft material that is impermeable to liquids, including blood or other physiological fluids.

In another example, the woven graft material may be made of a single material, or it may be a blend, weave, laminate, or composite of two or more materials. The graft material also may include other additives, such as plasticizers, compatibilizers, surface modifiers, biological materials such as peptides and enzymes, and therapeutic agents such as drugs or other pharmaceutically effective medicaments. The therapeutic agents can comprise agents, or combinations thereof, that can affect the cells in a vessel wall, including drugs, chromophores, and nucleic acids. Therapeutic agents also comprise diagnostics such as radiopaque compounds that allow the vessel to be visualized by fluoroscopy or like methods. Therapeutic agents can also comprise antimicrobial agents, such as antibacterial and antiviral agents.

It may be preferred that the socket includes a biocompatible polyurethane. Examples of biocompatible polyurethanes include Thoralon® (THORATEC, Pleasanton, Calif.), BIOSPAN®, BIONATE®, ELASTHANE®, PURSIL® and CARBOSIL® (POLYMER TECHNOLOGY GROUP, Berkeley, Calif.). As described in U.S. Pat. Pub. No. 2002/0065552 A1, incorporated herein by reference, Thoralon® is a polyetherurethane urea blended with a siloxane-containing surface modifying additive. Specifically, the polymer is a mixture of base polymer BPS-215 and an additive SMA-300. The concentration of additive may be in the range of 0.5% to 5% by weight of the base polymer. The BPS-215 component (THORATEC) is a segmented polyether urethane urea containing a soft segment and a hard segment. The soft segment is made of polytetramethylene oxide (PTMO), and the hard segment is made from the reaction of 4,4′-diphenylmethane diisocyanate (MDI) and ethylene diamine (ED). The SMA-300 component (THORATEC) is a polyurethane comprising polydimethylsiloxane as a soft segment and the reaction product of MDI and 1,4-butanediol as a hard segment. A process for synthesizing SMA-300 is described, for example, in U.S. Pat. Nos. 4,861,830 and 4,675,361, which are incorporated herein by reference. A polymer graft material may be formed from these two components by dissolving the base polymer and additive in a solvent such as dimethylacetamide (DMAC) and solidifying the mixture by solvent casting or by coagulation in a liquid that is a non-solvent for the base polymer and additive.

Thoralon® has been used in certain vascular applications and is characterized by thromboresistance, high tensile strength, low water absorption, low critical surface tension, and good flex life. Thoralon® is believed to be biostable and to be useful in vivo in long term blood contacting applications requiring biostability and leak resistance. Because of its flexibility, Thoralon® is useful in larger vessels, such as the abdominal aorta, where elasticity and compliance is beneficial.

Other polyurethane ureas may be used in addition to Thoralon. For example, the BPS-215 component with a MDI/PTMO mole ratio ranging from about 1.0 to about 2.5 may be used.

In addition to polyurethane ureas, other polyurethanes, preferably those having a chain extended with diols, may be used as the graft material. Polyurethanes modified with cationic, anionic, and aliphatic side chains also may be used. See, for example, U.S. Pat. No. 5,017,664, which is incorporated herein by reference. Polyurethanes may need to be dissolved in solvents such as dimethyl formamide, tetrahydrofuran, dimethyacetamide, dimethyl sulfoxide, or mixtures thereof.

The polyurethanes also may be end-capped with surface active end groups, such as, for example, polydimethylsiloxane, fluoropolymers, polyolefin, polyethylene oxide, or other suitable groups. See, for example, the surface active end groups disclosed in U.S. Pat. No. 5,589,563, which is incorporated herein by reference.

In one example, the graft material may contain a polyurethane having siloxane segments, also referred to as a siloxane-polyurethane. Examples of polyurethanes containing siloxane segments include polyether siloxane-polyurethanes, polycarbonate siloxane-polyurethanes, and siloxane-polyurethane ureas. Specifically, examples of siloxane-polyurethane include polymers such as ELAST-EON 2 and ELAST-EON 3 (AORTECH BIOMATERIALS, Victoria, Australia); polytetramethyleneoxide (PTMO) and polydimethylsiloxane (PDMS) polyether-based aromatic siloxane-polyurethanes such as PURSIL-10, 20, and -40 TSPU; PTMO and PDMS polyether-based aliphatic siloxane-polyurethanes such as PURSIL AL-5 and AL-10 TSPU; aliphatic, hydroxy-terminated polycarbonate and PDMS polycarbonate-based siloxane-polyurethanes such as CARBOSIL-10, -20, and -40 TSPU (all available from POLYMER TECHNOLOGY GROUP). The PURSIL, PURSIL-AL, and CARBOSIL polymers are thermoplastic elastomer urethane copolymers containing siloxane in the soft segment, and the percent siloxane in the copolymer is referred to in the grade name. For example, PURSIL-10 contains 10% siloxane. Examples of siloxane-polyurethanes are disclosed in U.S. Pat. Pub. No. 2002/0187288 A1, which is incorporated herein by reference.

The graft may contain polytetrafluoroethylene or ePTFE. The structure of ePTFE may be characterized as containing nodes connected by fibrils. The structure of ePTFE is disclosed, for example, in U.S. Pat. Nos. 6,547,815 B2; 5,980,799; and 3,953,566; all of which are incorporated herein by reference.

If so desired, the polymers described above may be processed to form porous polymer grafts using standard processing methods, including solvent-based processes such as casting, spraying, dipping, melt extrusion processes, repolymerization or thermoformation. Extractable pore forming agents may be used during processing to produce porous polymer graft material. Examples of the particulate used to form the pores include a salt, including, but not limited to, sodium chloride (NaCl), sodium bicarbonate (NaHCO₃), Na₂CO₃, MgCl₂, CaCO₃, calcium fluoride (CaF₂), magnesium sulfate (MgSO₄), CaCl₂, AgNO₃, or any water soluble salt. However, other suspended particulate materials may be used. These include, but are not limited to, sugars, polyvinyl alcohol, cellulose, gelatin, or polyvinyl pyrolidone. Preferably, the particulate is sodium chloride; more preferably, the particulate is a sugar.

Therapeutic agents may be incorporated into the graft material of the prosthesis, or into the biocompatible coating which encapsulates the stent, so that they may be released into the body surrounding the lumen wall upon expansion and curing of the prosthesis. Therapeutic agents or medicaments may be impregnated into the lumen wall by pressure from expansion of the prosthesis. The therapeutic agent can also be photoreleasably linked to the surface of the prosthesis so that, upon contact with the surrounding lumen wall, the agent is released onto the cells of the adjacent vascular wall by exposure to radiation delivered via an optical fiber.

The term “stent” means any device that provides rigidity, expansion force, or support to a prosthesis, such as a stent graft. In one configuration, the stent may represent a plurality of discontinuous devices. In another configuration, the stent may represent one device. The stent may be located on the exterior of the device, the interior of the device, or both. Stents may have a wide variety of configurations and may be balloon-expandable or self-expanding. Typically, stents have a circular cross-section when fully expanded, so as to conform to the generally circular cross-section of a body lumen. In one example, a stent may comprise struts and acute bends or apices that are arranged in a zig-zag configuration in which the struts are set at angles to each other and are connected by the acute bends. The stent struts may have a thickness ranging from about 0.060 mm to about 0.20 mm and all combinations and subcombinations therein.

Preferably, the stent is formed from nitinol, stainless steel, tantalum, titanium, gold, platinum, inconel, iridium, silver, tungsten, cobalt, chromium, or another biocompatible metal, or alloys of any of these. Examples of other materials that may be used to form stents include carbon or carbon fiber; cellulose acetate, cellulose nitrate, silicone, polyethylene teraphthalate, polyurethane, polyamide, polyester, polyorthoester, polyanhydride, polyether sulfone, polycarbonate, polypropylene, high molecular weight polyethylene, polytetrafluoroethylene, or another biocompatible polymeric material, or mixtures or copolymers of these; polylactic acid, polyglycolic acid or copolymers thereof; a polyanhydride, polycaprolactone, polyhydroxybutyrate valerate or another biodegradable polymer, or mixtures or copolymers of these; a protein, an extracellular matrix component, collagen, fibrin, or another biologic agent; or a suitable mixture of any of these. Preferably, the stent is a nitinol or stainless steel stent.

The socket may be comprised of biocompatible polyurethane, silicone infused polyurethane, such as Thoralon® (Thoratec, Pleasanton, Calif.), or Biospan®, Bionate®, Elasthane®, Pursil® And Carbosil® (Polymer Technology Group, Berkeley, Calif.). In some examples, polyurethane can also comprise SIS. The sockets may comprise a single biologically active material or a blend of materials that are thromboresistant. The sockets are thromboresistant without the addition of foams, adhesives, or polymers. In examples that may be preferred, the sockets are attached to the stent graft by reploymerization or they are thermoformed to the stent grafts.

Thoralon® has been used in certain vascular applications and is characterized by thromboresistance, high tensile strength, low water absorption, low critical surface tension, and good flex life. Thoralon® is believed to be biostable and to be useful in vivo in long term blood contacting applications requiring biostability and leak resistance. Because of its flexibility, Thoralone is useful in larger vessels, such as the abdominal aorta, where elasticity and compliance is beneficial.

As shown in FIG. 2, the stent graft has a stent graft wall 20 comprising a graft material for endoluminal implantation. As seen in FIG. 2, the stent graft wall 20 has an opening 25 that, once the stent graft is deployed, is intended to align with a branch vessel. At least one elastic, deformable socket 30 communicates with the opening 25. The socket 30 extends from the stent graft wall 20 of the stent graft with shock absorbing and elastic properties. The socket comprises a socket wall having a proximal end 23 and a distal end 29 with the proximal end 23 interconnected with the stent graft wall 20. The proximal end 23 of the socket 30 surrounds the opening 25 and the distal end 29 extends in a distal direction from the stent graft wall 20. The wall of the socket 30 at least partially encases a stent 27.

A secondary socket for receiving a secondary stent graft comprising an elastic wall forming a lumen also may be included. Some examples have at least one opening and at least one socket with the at least one socket being in communication with the openings. In some examples, that may be preferred, the stent graft further comprises a secondary socket that has no stent encased within the secondary socket wall. Such an example would have more than one socket attached to the stent graft having a stent encased within its walls and a secondary socket with no encased stent. Any of the sockets are capable of telescopically receiving a secondary stent graft.

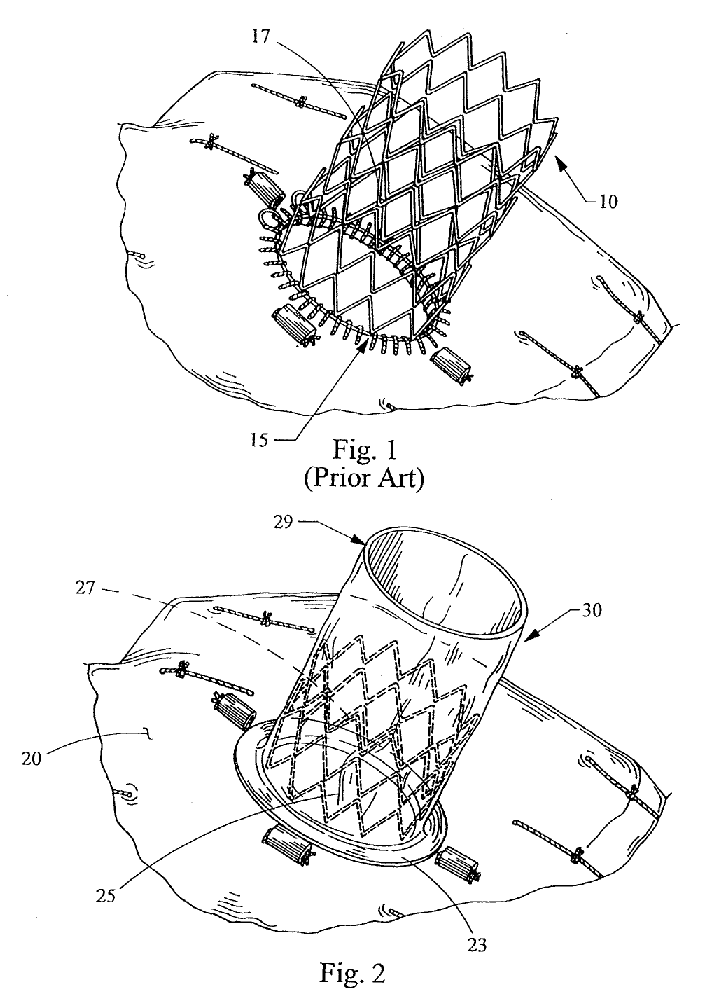

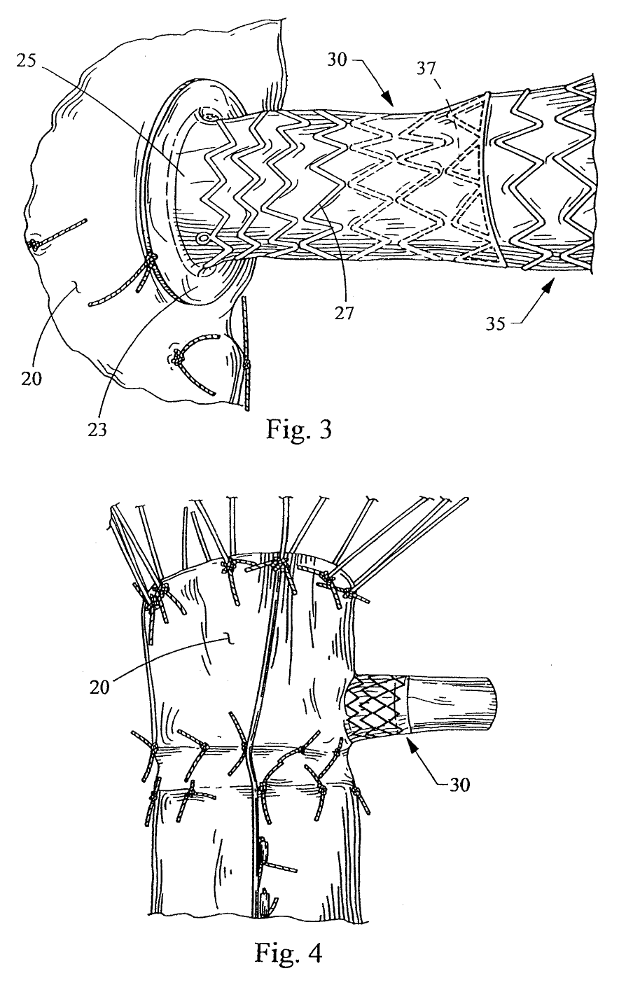

FIG. 1 is an illustration of a socket 10 commonly used in the prior art containing a stent 17 attached to a nitinol ring 15 sewn around the opening 25 in the graft. As seen in FIGS. 2 and 3, the proximal end 23 of the socket 30 of the present invention flares around the opening 25 in the stent graft wall 20. Although the proximal end of the socket is shown flared around the external side of the graft wall, the socket may flare around the internal side of the graft wall.

Due at least in part to its elastic attributes, the socket 30 has an expandable diameter. The socket 30 may be attached to the stent graft wall 20 by repolymerization. For instance, a solvent such as dimethylacetamide (DMAC) may be used to partially dissolve the polymer such that is penetrates into the graft material of the stent graft wall 20. The polymer is then allowed to repolymerize. Alternatively, the socket 30 may be glued to the stent graft wall 20. The polymer and DMAC may be used together in solution as a glue to attach the socket 30 and stent graft wall 20. In other examples, dipping or casting may be used to join the socket 30 and stent graft wall 20. Preferably, the means of attachment provides the socket 30 with a hermetic seal with the stent graft wall 20.

A branched prosthesis for implantation in an aortic arch 76 is shown in FIG. 10. The stent graft wall of the prosthesis 70 spans the aortic arch 76 and has two elastic, deformable sockets 82 deployed in the carotid 74 and subclavian 72 arteries. A stent graft 88 is shown deployed in the socket 82 within the subclavian artery 72. One socket may be included for each branch artery.

A compliant connection between a branched prosthesis and a stent or stent-graft deployed in the iliac or renal arteries also may be provided. This may help to lower the peak loads transferred through the interface and better accommodate the very dynamic nature of the operating environment.

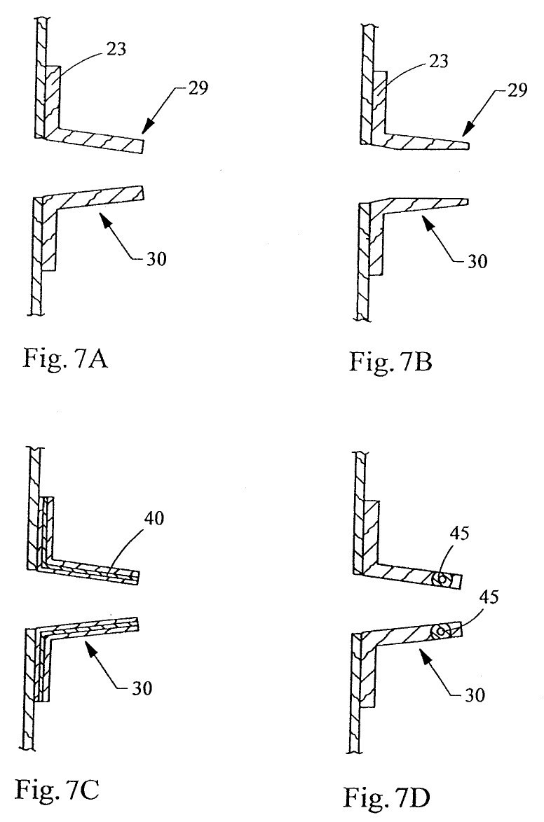

FIGS. 7A through 7D show cross-sectional views of the socket 30. In FIG. 7A, the illustration shows a cross-sectional view of a socket 30 tapering off to a diameter smaller at the distal end 29 than at the proximal end 23. Such a design may facilitate tracking during deployment and also help secure the stent and maintain sealing. This will also allow the radial stiffness of the socket 30 to vary along its length which will allow designs to span a wider variation in diameter without providing excessive force which may tend to crush other stents in the art.

The thickness and length of the socket 30 may vary, as in FIG. 7B, so that its radial stiffness may be controlled. The radial stiffness of the socket 30 may impact sealing and the pull out force. The socket 30 extends radially from the stent graft wall at an acute, right, or obtuse angle in varying examples. The socket 30 can comprise Thoralon which demonstrates significant elasticity (approximately 900%) so as to provide a wide range of operation. Alternatively, one skilled in the art can see a wide range of materials may be used herein, which includes ePTFE, polyurethane, and any other polymers which exhibit sufficient elasticity and/or deformability and, of course, biocompatibility.

Some examples of the socket 30 comprise reinforcing elements. These elements can comprise nitinol, stainless steel, or polyethylene fibers, for example. FIG. 7C shows a socket 30 encasing a reinforcing element 40. In some examples, the socket 30 comprises radiopaque markers 45. In the example illustrated in FIG. 7D, the markers 45 are in the distal end 29 of the socket 30. Gold markers, for instance, may be embedded within the socket 30 to ensure accurate deployment.

The socket 30 can have reinforcing elements, such as nitinol or PET fibers, imbedded to alter the radial and longitudinal stiffness of the socket 30, as shown in FIG. 7C. The resultant composite socket 30 can limit the range of its motion as a function of stent design. Therefore, the ultimate diameter of the socket 30 may be controlled to help prevent possible excessive vessel damage as the embedded reinforcing elements may be used to limit the expansion of the socket 30 during stent deployment. A stent may be a reinforcing element. Socket 30 stiffness may be adjusted to the different radial stiffness exhibited in self-expanding stents.

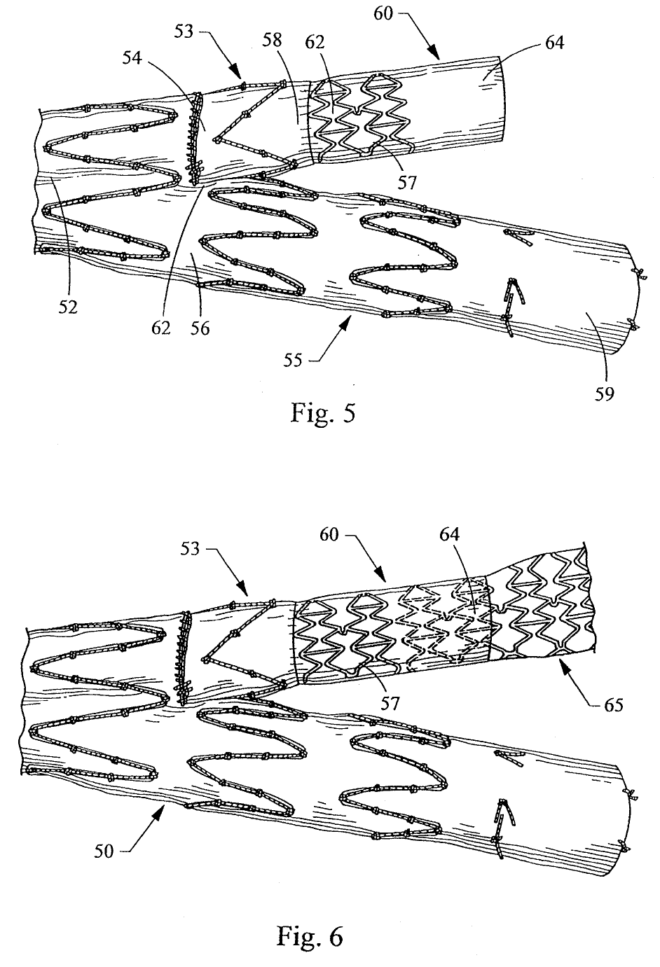

As illustrated in FIGS. 5 and 6, another example of the present invention provides a bifurcated stent graft 50 with two distal openings for deployment in the abdominal aorta. This stent graft 50 comprises a main section 52 that forms a main lumen configured for deployment in the aorta. There is a first branch section 53 and a second branch section 55 both having proximal portions 54, 56 and distal portions 58, 59. The proximal portion 54 of the first branch section 55 and the proximal portion 56 of the second branch section 55 meet with the main section 52 at the bifurcation 62. The first branch 53 and second branch 55 sections comprise a graft material and are configured for deployment in vessels arteries branching from aorta. The first branch 53 and second branch 55 sections forming a first lumen and a second lumen, respectively. The lumens are in fluid communication with the main lumen of the main section 52.

The bifurcated stent graft 50 further comprises an elastic, deformable socket 60 in communication with the opening at the distal portion 58 of the first branch section 53. The socket 60 comprises a socket wall that forms a socket lumen with the socket 60 having a proximal end 62 and a distal end 64. The proximal end 62 being attached to the graft material of a branch section at the distal portion of that branch section. Although the socket 60 shown is attached with the distal portion 58 of the first branch section 53, there are also examples not shown wherein a socket is attached with the distal portion 59 of the second branch 55 section. It is understood then that the opening of the stent graft in such examples is in the distal portions 58, 59.

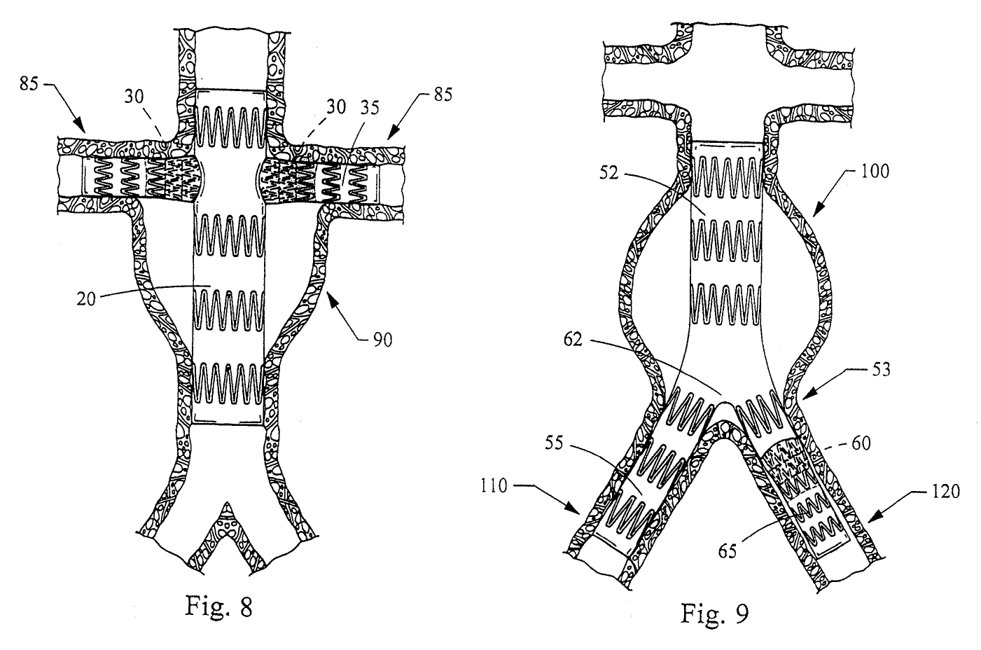

The elastic, deformable sockets of the present invention are configured to be receptive to tubular prostheses suitable for deployment in branch vessels. FIG. 3 is a close-up view of a secondary stent graft 35 deployed in the distal end of the deformable socket 30 in one particular example. The stents 37 of the secondary stent graft 35 are indicated with dashed markings. Such an example is suitable for implantation in an abdominal aortic aneurysm (AAA) 90 with two deformable sockets 30 acting as bridges to the renal arteries 85. A secondary stent graft 35 is deployed in a renal artery 85 as shown in FIG. 8.

In FIG. 6, a stent graft 65 is deployed within the socket 60 on a bifurcated prosthesis. Such an example is suitable for deployment in an AAA 100 as shown in FIG. 9. The main section 52 of the bifurcated stent graft is deployed in the aorta to occlude the aneurysm 100, while the second branch section 55 is deployed in the common illiac artery 110. The first branch section 53 may be deployed after the main section 52 and second branch section 53 have been secured in the AAA 100. In the example shown, a stent graft 65 is deployed and secured in the deformable socket 60. There are also examples comprising a socket proximal to the bifurcation with the socket comprising an elastic wall forming a lumen with a stent at least partially encased within the wall.

The sockets provided by the present invention have a variety of shapes for endovascular treatment or repair. The prostheses of the present invention can comprise sockets wherein the diameter is expandable up to about 10% of the socket's predeployment diameter. The socket can extend radially from the tubular prosthesis up to about 50 mm. In some examples, the socket extends radially from the tubular prosthesis for about 10 mm to about 40 mm.

In some examples of the present invention the stent graft, or tubular graft, for endoluminal implantation comprises an evertible elastic socket 130 comprising a socket wall, a first end 120, a second end 135, and a lumen therethrough, an everted configuration and extended configuration, where the first end 120 of the socket 130 is disposed adjacent the opening 125 of the tubular graft in the everted and extended configurations, and the second end 135 is disposed in the lumen of the graft in the everted configuration and is disposed external to the graft in the expanded configuration. In other examples, there is at least one evertible elastic socket 130 in fluid communication with at least one opening 125 in the stent graft. There are also examples (not shown), wherein the tubular graft has two openings and there are two evertible elastic grafts corresponding to the two openings.

In another example, there is an elastic socket 130 comprising a socket wall, a first end 120, a second end 135, and a stent 137 at least partially encased within the socket wall. The elastic socket 130 has an everted configuration and an extended configuration where the socket 130 is in fluid communication with the opening 125 in both the everted and extended configurations. The second end 135 is disposed in the lumen of the tubular graft in the everted configuration and is disposed external to the tubular graft in the expanded configuration.

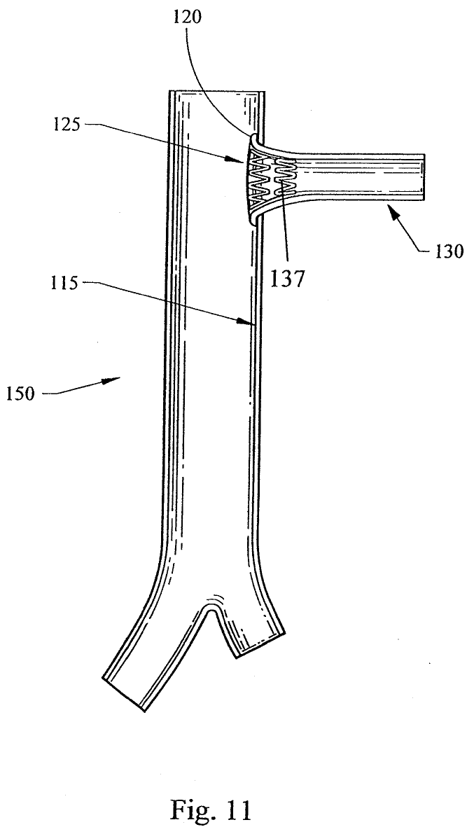

As shown in FIG. 11, the first end 120 of the socket 130 flares around the opening 125 of the interior of the tubular graft wall 115. The second end 135 of the socket 130 is within the lumen such that it may be everted through the opening 125. The elasticity of the socket 130 allows for this evertibility. As in other examples of this invention, the socket 130 at least partially encases a stent 127.

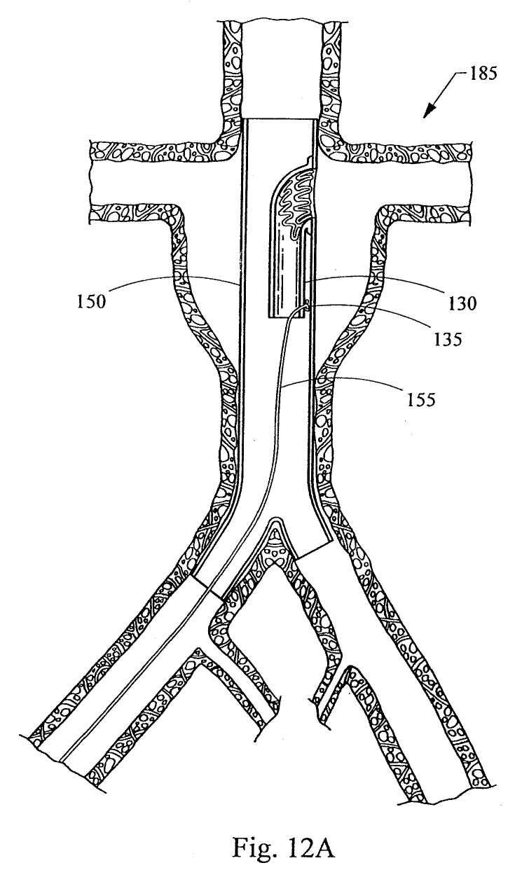

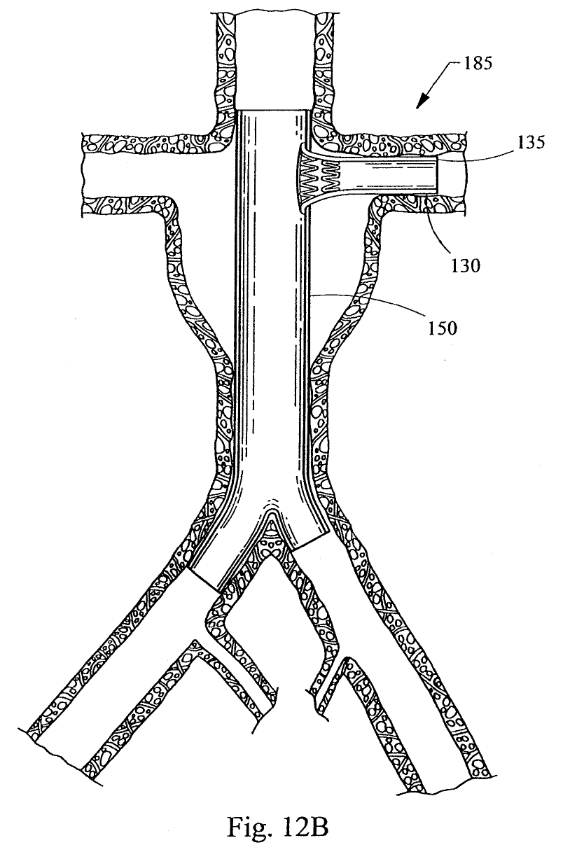

FIG. 12A shows an expanded view of the evertible socket 130 in a bifurcated stent graft 150. A guidewire 155 is attached to the second end 135 of the socket 130 so that it may be advanced into the renal artery 185. When the guidewire 155 is advanced, the socket 130 is everted into the renal artery 185 as shown in FIG. 12B.

In accordance with this example, there is a method of delivering a stent graft. The method comprises placing the tubular graft into a vessel having a branch artery such as the aorta. The opening 125 in the tubular graft wall 115 is aligned with the branch artery for eventual deployment of the socket 130. A guidewire 155 is pulled into the branch artery to evert the elastic socket 130 and to deploy the elastic socket 130 into the branch artery. The guidewire 155 is moved through the lumen of the elastic socket 130 to bring the second end of the elastic socket 130 into the lumen of the branch artery.

Throughout this specification, various indications have been given as to preferred and alternative examples of the invention. However, it should be understood that the invention is not limited to any one of these. It is therefore intended that the foregoing detailed description be regarded as illustrative rather than limiting, and that it be understood that it is the appended claims, including all equivalents, that are intended to define the spirit and scope of this invention.

1. An endoluminal prosthesis comprising:
a tubular graft having a proximal end, a distal end, a lumen therethrough, and an opening between the proximal end and distal end; and an evertible elastic socket disposed within the lumen of the tubular graft,
the elastic socket comprising a socket wall, a first end, a second end, and a lumen therethrough, an everted configuration and extended configuration, where the first end of the socket is disposed adjacent the opening of the tubular graft in the everted and extended configurations, and the second end is disposed in the lumen of the graft in the everted configuration and is disposed external to the graft in the expanded configuration.

2. The endoluminal prosthesis of claim 1 wherein the elastic socket comprises a stent at least partially encased within the socket wall.

3. The endoluminal prosthesis of claim 1 wherein the second end of the elastic socket flares about the opening.

4. The endoluminal prosthesis of claim 1 wherein the elastic socket further comprises nitinol or polyethylene fibers.

5. The endoluminal prosthesis of claim 1 wherein the elastic socket is attached to the stent graft by repolymerization or is thermoformed.

6. The endoluminal prosthesis of claim 1 wherein the elastic socket is tapered.

7. The endoluminal prosthesis of claim 1 wherein the elastic socket comprises radiopaque markers.

8. The endoluminal prosthesis of claim 1 wherein the elastic socket is comprised of polyurethane or ePTFE.

9. The endoluminal prosthesis of claim 1 wherein the elastic socket comprises graft material.

10. The endoluminal prosthesis of claim 1 wherein the elastic socket has an expandable diameter.

11. The endoluminal prosthesis of claim 1 wherein the elastic socket is adapted for receiving a secondary stent graft.

12. The endoluminal prosthesis of claim 1 further comprising more than one opening and more than one elastic socket that corresponds to the more than one opening.

13. The endoluminal prosthesis of claim 1 further comprising a guidewire coupled to the elastic socket for everting the elastic socket out of the primary lumen and into a branch artery.

14. The endoluminal prosthesis of claim 1 wherein the elastic socket is adapted to telescopically receive a secondary prosthesis while placed in a branch artery.

15. The endoluminal prosthesis of claim 1 further comprising a reinforcing ring around the opening.

16. An endoluminal prosthesis comprising:
a tubular graft having a lumen therethrough, an opening, and an evertible elastic socket disposed within the lumen of the tubular graft,
the elastic socket comprising a socket wall, a first end, a second end, a stent at least partially encased within the socket wall, an everted configuration and an extended configuration, where the socket is in fluid communication with the opening in the everted and extended configurations, and the second end is disposed in the lumen of the tubular graft in the everted configuration and is disposed external to the tubular graft in the expanded configuration.

17. A method of delivering an endoluminal prosthesis comprising a tubular graft with a lumen therethrough, an opening between a proximal and distal end, and an evertible, elastic socket coupled to a guidewire, the socket comprising a first end, a second end, and a socket wall forming a lumen in fluid communication with the opening, the method comprising:
placing the tubular graft in a vessel having a branch artery;
aligning the opening with the branch artery; and
actuating the guidewire to evert the elastic socket and to deploy the elastic socket into the branch artery.

18. The method of claim 17 further comprising deploying a secondary stent graft into the elastic socket.

19. The method of claim 17 wherein the first end of the elastic socket is disposed adjacent the opening of the tubular graft, the second end is disposed in the lumen of the graft during the placing step, and the guidewire is coupled to the second end of the elastic socket.

20. A method of delivering an endoluminal prosthesis comprising a tubular graft with a lumen therethrough, an opening between a proximal and distal end, and an evertible, elastic socket coupled to a guidewire, the socket comprising a first end, a second end, and a socket wall forming a lumen in fluid communication with the opening, and a stent at least partially encased within the socket wall, the method comprising:
placing the tubular graft in a vessel having a branch artery;
aligning the opening with the branch artery; and
actuating the guidewire to evert the elastic socket and to deploy the elastic socket into the branch artery.
